(19) United States
(12) Patent Application Publication
(10) Pub. No.: US 2007/0249603 A1
(43) Pub. Date: Oct. 25, 2007

(54) 3-Arylsulfonyl-Quinolines as 5-Ht6 Receptor Antagonists for the Treatment of Cns Disorders

(76) Inventors: Christopher Norbert Johnson, Harlow Essex (GB); Geoffrey Stemp, Harlow Essex (GB); Mervyn Thompson, Harlow Essex (GB); David R. Witty, Harlow (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/569,383

(22) PCT Filed: May 19, 2005
(86) PCT No.: PCT/EP05/05585

§ 371 Date: Nov. 20, 2006

(30) Foreign Application Priority Data
May 21, 2004 (GB) 0411421.1

(51) Int. Cl. A61K 31/47; A61K 31/4725; A61K 31/5377; C07D 215/18; C07D 215/36; C07D 413/00

(52) U.S. Cl. 514/235.2; 514/311; 514/312; 544/128; 546/172; 546/180

(57) ABSTRACT

The present invention relates to novel quinoline derivatives such as compounds of the formula (I) which have antagonist potency for the 5-HT₆ receptor: 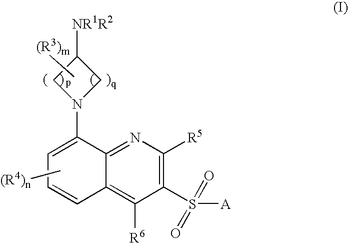 and the use of such compounds or pharmaceutical compositions thereof in the treatment of CNS and other disorders.

This invention relates to novel quinoline compounds having pharmacological activity, to processes for their preparation, to compositions containing them and to their use in the treatment of CNS and other disorders.

WO03/080580 (Glaxo Group Limited) describes a series of quinolinyl derivatives as compounds which possess affinity for the 5-HT₆ receptor. JP 02262627 (Japan Synthetic Rubber Co) describes a series of substituted quinoline derivatives useful as wavelength converting elements. WO 00/42026 (Novo Nordisk) describes a series of quinoline and quinoxaline compounds for use as GLP-1 agonists. WO 04/000828 (Biovitrum AB) describe a series of bicyclic sulfone or sulfonamide compounds which are claimed to be useful in the treatment or prophylaxis of a 5-HT₆ receptor related disorder. WO 00/71517 describes a series of phenoxypropylamine compounds as 5-HT_{1A} receptor antagonists which are claimed to be useful as anti-depressants.

A structurally novel class of compounds has now been found which also possess antagonist potency for the 5-HT₆ receptor. The present invention therefore provides, in a first aspect, a compound of formula (I) or a pharmaceutically acceptable salt thereof: 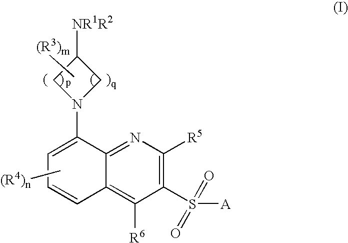
wherein:
R¹ and R² independently represent hydrogen or C₁₋₆ alkyl or R¹ and R² together with the nitrogen atom to which they are attached form a nitrogen containing heterocyclyl group optionally substituted by one or more halogen or C₁₋₆ alkyl groups;
p and q independently represent an integer from 1 to 3;
R³ represents C₁₋₄ alkyl;
m represents an integer from 0 to 4;
R⁴ represents halogen, cyano, —CF₃, CF₃O—, C₁₋₆ alkyl, C₁₋₆ alkoxy, C₁₋₆ alkanoyl or a group —CONR⁷R⁸;
n represents 0 to 3;
R⁵ and R⁶ independently represent hydrogen, halogen, cyano, —CF₃, CF₃O—, C₁₋₆ alkyl, C₁₋₆ alkoxy, C₁₋₆ alkanoyl or a group —CONR⁷R⁸;
R⁷ and R⁸ independently represent hydrogen or C₁₋₆ alkyl or together with the nitrogen atom to which they are attached form a nitrogen containing heterocyclyl or nitrogen containing heteroaryl group;
A represents an -aryl, -heteroaryl, -aryl-aryl, -aryl-heteroaryl, -heteroaryl-aryl or -heteroaryl-heteroaryl group;
   wherein said aryl and heteroaryl groups of A may be optionally substituted by one or more (eg. 1, 2 or 3) substituents which may be the same or different, and which are selected from the group consisting of halogen, hydroxy, cyano, nitro, trifluoromethyl, trifluoromethoxy, C₁₋₆ alkyl, trifluoromethanesulfonyloxy, pentafluoroethyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, arylsulfonyl, arylsulfonyloxy, arylsulfonylC₁₋₆ alkyl, C₁₋₆ alkylsulfonamido, C₁₋₆ alkylamido, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, aroylC₁₋₆ alkyl, arylC₁₋₆ alkanoyl, or a group —CONR⁹R¹⁰ or —SO₂NR⁹R¹⁰, wherein R⁹ and R¹⁰ independently represent hydrogen or C₁₋₆ alkyl or R⁹ and R¹⁰ together with the nitrogen atom to which they are attached may form a nitrogen containing heterocyclyl or nitrogen containing heteroaryl group; or solvates thereof.

Alkyl groups, whether alone or as part of another group, may be straight chain or branched and the groups alkoxy and alkanoyl shall be interpreted similarly. Alkyl moieties are more preferably C₁₋₄ alkyl, eg. methyl or ethyl.

The term ‘cycloalkyl’ unless otherwise stated means a closed 3- to 8-membered non-aromatic ring, for example cyclobutyl, cyclopentyl, cyclohexyl or cycloheptyl, or cyclooctyl.

The term ‘halogen’ is used herein to describe, unless otherwise stated, a group selected from fluorine, chlorine, bromine or iodine.

The term “aryl” includes single and fused rings for example, phenyl or naphthyl.

The term “heteroaryl” is intended to mean a 5 to 7 membered monocyclic aromatic or a fused 8 to 10 membered bicyclic aromatic ring containing 1 to 3 heteroatoms selected from oxygen, nitrogen and sulfur. Suitable examples of such monocyclic aromatic rings include thienyl, furyl, pyrrolyl, triazolyl, imidazolyl, oxazolyl, thiazolyl, oxadiazolyl, isothiazolyl, isoxazolyl, thiadiazolyl, pyrazolyl, pyrimidyl, pyridazinyl, pyrazinyl and pyridyl. Suitable examples of such fused bicyclic aromatic rings include benzofused aromatic rings such as quinolinyl, isoquinolinyl, quinazolinyl, quinoxalinyl, cinnolinyl, naphthyridinyl, indolyl, indazolyl, pyrrolopyridinyl, benzofuranyl, benzothienyl, benzimidazolyl, benzoxazolyl, benzisoxazolyl, benzothiazolyl, benzisothiazolyl, benzoxadiazolyl, benzothiadiazolyl and the like. Heteroaryl groups, as described above, may be linked to the remainder of the molecule via a carbon atom or, when present, a suitable nitrogen atom except where otherwise indicated above.

The term “nitrogen containing heteroaryl” is intended to represent any heteroaryl group as defined above which contains a nitrogen atom.

It will be appreciated that wherein the above mentioned aryl or heteroaryl groups have more than one substituent, said substituents may be linked to form a ring, for example a carboxyl and amine group may be linked to form an amide group.

The term “heterocyclyl” is intended to mean a 4 to 7 membered monocyclic saturated or partially unsaturated aliphatic ring containing 1 to 3 hetroatoms selected from oxygen, nitrogen or sulphur; or a 4 to 7 membered monocyclic saturated or partially unsaturated aliphatic ring containing 1 to 3 heteroatoms selected from oxygen, nitrogen or sulfur fused to a benzene or monocyclic heteroaryl ring (referred to as fused rings). Suitable examples of such monocyclic rings include pyrrolidinyl, piperidinyl, piperazinyl, morpholinyl, thiamorpholinyl, diazepanyl, azepanyl, dihydroimidazolyl, tetrahydropyranyl, tetrahydrothiapyranyl and tetrahydrofuranyl. Suitable examples of fused rings include dihydroindolyl, dihydroisoindolyl, tetrahydroquinolinyl, tetrahydrobenzazepinyl and tetrahydroisoquinolinyl.

The term “nitrogen containing heterocyclyl” is intended to represent any heterocyclyl group as defined above which contains a nitrogen atom.

In one embodiment, R¹ and R² independently represent hydrogen or C₁₋₆ alkyl or R¹ and R² together with the nitrogen atom to which they are attached form a nitrogen containing heterocyclyl group optionally substituted by 1 to 3 halogen or C₁₋₆ alkyl groups. In one embodiment, R¹ and R² independently represent hydrogen or C₁₋₆ alkyl (e.g. methyl, ethyl or isopropyl) or R¹ and R² together with the nitrogen atom to which they are attached form a pyrrolidinyl, piperidinyl, or morpholinyl ring.

In one embodiment, R¹ and R² both represent C₁₋₆ alkyl (eg. methyl or ethyl) or R¹ and R² together with the nitrogen atom to which they are attached form a pyrrolidinyl, piperidinyl, or morpholinyl ring optionally substituted by one or more halogen (eg. fluorine) groups.

In one embodiment, p and q both represent 1 or 2 or one of p and q represents 1 and the other represents 2.

In one embodiment, m represents zero.

In one embodiment, R⁴ represents halogen, for example iodine.

In one embodiment, n represents zero.

In one embodiment, R⁵ and R⁶ both represent hydrogen.

In one embodiment, A represents -aryl (eg. phenyl) optionally substituted by one or more halogen (eg. chlorine) atoms or -heteroaryl (eg. pyridyl). In a further embodiment A represents -aryl (eg. phenyl) optionally substituted by a halogen (eg. chlorine). In yet a further embodiment A represents unsubstituted phenyl.

In one embodiment, A represents phenyl optionally substituted by one or more halogen atoms.

In a further embodiment there is provided a compound of formula (Ia) or a pharmaceutically acceptable salt thereof: 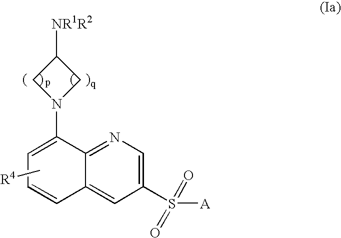
wherein:
R¹ and R² independently represent hydrogen or C₁₋₆alkyl (e.g. methyl, ethyl or isopropyl) or R¹ and R² together with the nitrogen atom to which they are attached form a pyrrolidinyl, piperidinyl, or morpholinyl ring;
p and q both represent 1 or 2 or one of p and q represents 1 and the other represents 2;
R⁴ represents hydrogen or halogen; and
A represents phenyl optionally substituted by one or more halogen atoms.

Preferred compounds according to the invention include examples E1-E13 as shown below, or a pharmaceutically acceptable salt thereof.

The compounds of formula (I) can form acid addition salts thereof. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. The present invention includes within its scope all possible stoichiometric and non-stoichiometric forms.

The compounds of formula (I) may be prepared in crystalline or non-crystalline form, and, if crystalline, may optionally be solvated, eg. as the hydrate. This invention includes within its scope stoichiometric solvates (eg. hydrates) as well as compounds containing variable amounts of solvent (eg. water).

Certain compounds of formula (I) are capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The present invention also provides a process for the preparation of a compound of formula (I) or a pharmaceutically acceptable salt thereof, which process comprises:
(a) preparing a compound of formula (I) which comprises reacting a compound of formula (II) 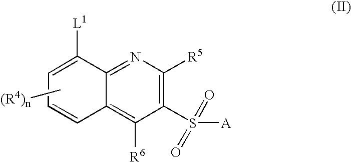
   or an optionally protected derivative thereof, wherein R⁴, R⁵, R⁶, n and A are as defined above and L¹ represents a suitable leaving group such as a halogen atom or a trifluoromethylsulfonyloxy group, with a compound of formula (III) 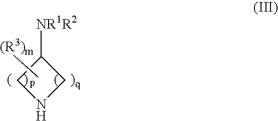
   or an optionally protected derivative thereof, wherein R¹, R², R³, m, p and q are as defined above; and optionally thereafter
(b) deprotecting a compound of formula (I) which is protected;
(c) interconversion to other compounds of formula (I) and/or forming a pharmaceutically acceptable salt and/or solvate.

Process (a) typically comprises the use of basic conditions and may be conveniently carried out using a compound of formula (II) wherein L¹ represents a fluorine atom and a compound of formula (III) in a suitable solvent such as dimethyl sulfoxide in the presence of a suitable base such as anhydrous potassium carbonate. Process (a) may be optionally carried out at elevated temperature, e.g. 90-110° C.

In processes (a) and (b) examples of protecting groups and the means for their removal can be found in T. W. Greene ‘Protective Groups in Organic Synthesis’ (J. Wiley and Sons, 1991). Suitable amine protecting groups include sulphonyl (e.g. tosyl), acyl (e.g. acetyl, 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrochloric acid) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid. A further amine protecting group includes methyl which may be removed using standard methods for N-dealkylation (e.g. 1-chloroethyl chloroformate under basic conditions followed by treatment with methanol).

Process (c) may be performed using conventional interconversion procedures such as epimerisation, oxidation, reduction, reductive alkylation, alkylation, nucleophilic or electrophilic aromatic substitution, ester hydrolysis or amide bond formation. For example, N-dealkylation of a compound of formula (I) wherein R¹ or R² represents an alkyl group to give a compound of formula (I) wherein R¹ or R² represents hydrogen. It will be appreciated that such interconversion may be interconversion of protected derivatives of formula (I) which may subsequently be deprotected following interconversion.

In addition, process (c) may also comprise, for example, reacting a compound of formula (I), wherein R¹ or R² represents hydrogen, with an aldehyde or ketone in the presence of a reducing agent in order to generate a compound of formula (I) where R¹ or R² represents C₁₋₆alkyl. This may be performed using a hydride donor agent such as sodium cyanoborohydride, sodium triacetoxyborohydride or a resin bound form of cyanoborohydride in an alcoholic solvent such as ethanol and in the presence of an acid such as acetic acid, or under conditions of catalytic hydrogenation. Alternatively, such a transformation may be carried out by reacting a compound of formula (I), wherein R¹ or R² represents hydrogen, with a compound of formula R^{1a}—L or R^{2a}—L, wherein R^{1a} and R^{2a} represent C₁₋₆-alkyl and L represents a leaving group such as a halogen atom (e.g. bromine or iodine) or methylsulfonyloxy group, optionally in the presence of a suitable base such as potassium carbonate or triethylamine using an appropriate solvent such as N,N-dimethylformamide or a C₁₋₄alkanol.

Compounds of formula (II) may be prepared as described in WO 2003/080580.

Compounds of formula (II) wherein L¹ represents a fluorine or chlorine atom may also be prepared by reacting a compound of formula (IV) 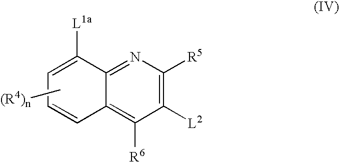
wherein R⁴, R⁵, R⁶ and n are as defined above, L^{1a} is a fluorine or chlorine atom and L² is a suitable leaving group such as an iodine atom; with a compound of formula A—SO₂—M, wherein A is as defined above and M is a metal residue such as sodium or potassium, in the presence of a copper (I) salt, e.g. copper (I) triflate or copper (I) iodide, in a suitable solvent such as dimethyl sulfoxide, anhydrous N,N-dimethylformamide or 1,4-dioxane, optionally including a ligand such as N,N′-dimethyl-ethylene-1,2-diamine and optionally in the presence of a base such as potassium carbonate.

Compounds of formula (IV) wherein L² represents an iodine atom may be prepared from compounds of formula (V) 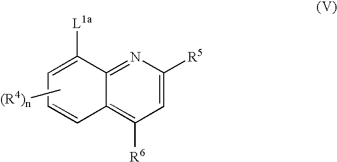
wherein L^{1a}, R⁴, R⁵, R⁶ and n are as defined above; with an iodinating agent such as N-iodosuccinimide in a suitable solvent such as acetic acid.

Compounds of formula (III) and (V) are either known in the literature or may be prepared by analogous methods.

Pharmaceutically acceptable salts may be prepared conventionally by reaction with the appropriate acid or acid derivative.

Compounds of formula (I) and their pharmaceutically acceptable salts have affinity for the 5-HT₆ receptor and are believed to be of potential use in the treatment of certain CNS disorders such as anxiety, depression, epilepsy, obsessive compulsive disorders, migraine, cognitive memory disorders (e.g. Alzheimers disease, age related cognitive decline, mild cognitive impairment and vascular dementia), Parkinsons Disease, ADHD (Attention Deficit Disorder/Hyperactivity Syndrome), sleep disorders (including disturbances of Circadian rhythm), feeding disorders such as anorexia and bulimia, panic attacks, withdrawal from drug abuse such as cocaine, ethanol, nicotine and benzodiazepines, schizophrenia (in particular cognitive deficits of schizophrenia), stroke and also disorders associated with spinal trauma and/or head injury such as hydrocephalus. Compounds of the invention are also expected to be of use in the treatment of certain GI (gastrointestinal) disorders such as IBS (Irritable Bowel Syndrome). Compounds of the invention are also expected to be of use in the treatment of obesity.

Thus the invention also provides a compound of formula (I) or a pharmaceutically acceptable salt thereof, for use as a therapeutic substance, in particular in the treatment or prophylaxis of the above disorders. In particular the invention provides for a compound of formula (I) or a pharmaceutically acceptable salt thereof, for use in the treatment of depression, anxiety, Alzheimers disease, age related cognitive decline, ADHD, obesity, mild cognitive impairment, schizophrenia, cognitive deficits in schizophrenia and stroke.

The invention further provides a method of treatment or prophylaxis of the above disorders, in mammals including humans, which comprises administering to the sufferer a therapeutically effective amount of a compound of formula (I) or a pharmaceutically acceptable salt thereof.

In another aspect, the invention provides the use of a compound of formula (I) or a pharmaceutically acceptable salt thereof in the manufacture of a medicament for use in the treatment or prophylaxis of the above disorders.

5-HT₆ antagonists have the potential to be capable of increasing basal and learning-induced polysialylated neuron cell frequency in brain regions such as the rat medial temporal lobe and associated hippocampus, as described in WO 03/066056. Thus, according to a further aspect of the present invention, we provide a method of promoting neuronal growth within the central nervous system of a mammal which comprises the step of administering a compound of formula (I) or a pharmaceutically acceptable salt thereof.

In order to use the compounds of formula (I) in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof, and a pharmaceutically acceptable carrier.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusable solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 0.05 to 200 mg, for example 20 to 40 mg; and such unit doses will preferably be administered once a day, although administration more than once a day may be required; and such therapy may extend for a number of weeks or months.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention.

List of Abbreviations Used:

AcOH: acetic acid

DMSO: dimethyl sulphoxide

RT: room temperature

HPLC: high performance liquid chromatography

### Description 1

### 8-Fluoro-3-iodoquinoline (D1)

N-Iodosuccinimide (8.1 g, 36.0 mmol, 2 eq.) was added to a solution of 8-fluoroquinoline (2.65 g, 18.0 mmol) in AcOH (13.25 ml, 5 vol). The mixture was stirred and placed in an oil bath which was then heated to 80° C. After 20 hrs 25 min the flask was removed from the oil bath and allowed to cool to room temperature. Dichloromethane (13.5 ml) was added, the solution was washed with 10% w/v Na₂SO_{3(aq)} (23.5 ml), then with H₂O (13.5 ml) before being concentrated under reduced pressure. The crude product was pre-absorbed on silica and purified via column chromatography, eluting with 19:1 isohexane/EtOAc 1% Et₃N to yield 8-fluoro-3-iodoquinoline (D1) as a white solid (3.46 g, 12.7 mmol, 70%).

¹H NMR (CDCl₃, 400M Hz) δ7.40-7.45 (1H, m, ArH), 7.50-7.52 (2H, m, ArH), 8.58 (1H, t, J 1.7 Hz, ArH), 9.09 (1 H, d, J 2.0 Hz, ArH).

### Description 2

### 8-Fluoro-3-(phenylsulfonyl)quinoline (D2)

A flask was charged with CuI (70 mg, 0.366 mmol, 0.1 eq.), 8-fluoro-3-iodoquinoline (D1) (1.00 g, 3.66 mmol), benzenesulfinic acid sodium salt (1.56 g, 10.98 mmol, 3 eq.) and potassium carbonate (1.01 g, 7.32 mmol, 2eq). DMSO (5 ml, 5 vol.) then N, N′-dimethylethylenediamine (0.078 ml, 0.2 eq.) were added, the mixture was stirred and placed in an oil bath which was heated to 90° C. After heating for 3½ hrs the flask was removed from the oil bath and allowed to cool to room temperature. The mixture was filtered and the cake was washed with DMSO (2×2 ml), the cake was then slurried with water (4 ml) and filtered, then washed with water (2×2 ml), sucked dry and further dried in at 50° C. under reduced pressure to yield 8-fluoro-3-(phenylsulfonyl)quinoline (D2) as an off-white solid (0.485 g, 46%)

¹H NMR (CDCl₃, 400 MHz) δ7.54-7.67 (5H, m, ArH), 7.78 (1 H, d, J 8.3 Hz, ArH), 8.04 (2H, m, ArH), 8.85 (1H, m, ArH), 9.31 (1H, d, J 2.0 Hz, ArH).

### Description 3

### 8-Chloro-3-iodoquinoline (D3)

N-Iodosuccinimide (206.3 g, 0.92 mol) was added portionwise over 1 h to a stirred solution of 8-chloroquinoline (150 g, 0.92 mol) in acetic acid (750 ml) at 40° C. The reaction temperature was then increased to 65° C. and this was maintained for 18 h after which another portion of N-iodosuccinimide (61.9 g, 0.28 mmol) was added. After a further 4 h at this temperature, the mixture was cooled to ambient temperature and evaporated in vacuo to an oil. The oil was dissolved in dichloromethane (600 ml) and the solution was washed with saturated sodium thiosulfate solution (2×400 ml), dried (MgSO₄) and concentrated in vacuo to a solid (280 g). The solid was recrystallized from ethyl acetate (300 ml) to afford the title compound (D3) as a yellow solid (80 g). Concentration of the corresponding filtrate gave a second crop of title compound (30 g, total yield 45%).

MS: m/z (M+H)⁺ C₉H₅CIIN requires 289, 291; found 290, 292 (MH⁺).

### Description 4

### 8-Chloro-3-[(4-fluorophenyl)thio]quinoline (D4)

Successive portionwise additions of potassium phosphate (102.7 g, 0.48 mol), copper (I) iodide (2.3 g, 12 mmol) and 8-chloro-3-iodoquinoline (D3) (70 g, 0.24 mol) were added with stirring to ethylene glycol (1 L) at ambient temperature. 4-Fluorobenzenethiol (38.6 ml, 0.363 mol) was added to the mixture in one portion and the whole was heated with stirring at 80° C. for 18 h. The mixture was then cooled to ambient temperature and water (800 ml) and dichloromethane (800 ml) were added. After vigorously stirring for 20 mins, the layers were separated and the stirred organic phase was treated with charcoal (20 g). After 0.5 h stirring, the mixture was filtered and the filtrate washed with water (500 ml), dried and concentrated in vacuo to afford 8-chloro-3-[(4-fluorophenyl)thio]quinoline (D4) as a crude yellow solid (78 g, 0.27 mol, 100%) which was used without purification in the next stage (see D5).

MS: m/z (M+H⁺) 290, 292; C₁₅H₉CIFNS requires 289, 291.

### Description 5

### 8-Chloro-3-[(4-fluorophenyl)sulfonyl]quinoline (D5)

A solution of 8-chloro-3-[(4-fluorophenyl)thio]quinoline (D4) (70 g, nominal value 0.242 mol) in dichloromethane (200 ml) was added dropwise to a stirred mixture of hydrated monomagnesium peroxyphthalate (270 g, 0.545 mol) in dichloromethane (800 ml) and methanol (200 ml) at 0° C. After completed addition, the mixture was stirred for 48 h at ambient temperature. To this mixture was slowly added a 10% solution of sodium sulfite (500 ml) and the temperature kept below 30° C. whilst stirring for 0.5 h. The layers were separated and the organic phase was washed with saturated sodium hydrogen carbonate solution (2×300 ml) and concentrated in vacuo to a volume of approximately 300 ml. After cooling this solution in ice, the precipitated solid was filtered, washed with cold dichloromethane (200 ml), dried in vacuo at 35° C. for 12 h and identified as the title compound (D5)(30 g, 93.5 mmol, 39%).

MS: m/z (M+H)⁺ 322, 324; C₁₅H₉CIFNO₂S requires 321, 323.

### EXAMPLE 1a

### 3-Phenylsulfonyl-8-[(4-dimethylamino)-piperidin-1-yl]-quinoline (E1a)

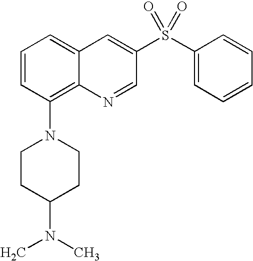

A solution of 8-fluoro-3-(phenylsulfonyl)quinoline (D2) (287 mg, 1.0 mmol) and 4-dimethylamino-piperidine (770 mg, 5.0 mmol) in DMSO was stirred with potassium carbonate (276 mg, 2.0 mmol) in DMSO (2 ml) at 100° C. for 16 h under an argon atmosphere. The mixture was cooled to RT, poured into water (50 ml) and extracted with ethyl acetate (50 ml). The organic phase was then washed a further three times with water, dried, (anhydrous sodium sulfate) and concentrated in vacuo to afford the 3-phenylsulfonyl-8-[(4-dimethylamino)-piperidin-1-yl]-quinoline (E1a) (413 mg, 0.95 mmol, 95%) as a yellow solid.

¹H NMR (CDCl₃) δ1.85-2.01 (4H, m), 2.35 (6H, s), 2.38 (1H, m), 2.73-2.86 (2H, m), 3.90-4.02 (2H, m), 7.29 (1 H, d, J=2.6 Hz), 7.45-7.62 (5H, m), 7.98-8.06 (2H, m), 8.76 (1 H, d, J=2.6 Hz), 9.22 (1 H, d, J=2.6 Hz).

MS: m/z (M+H)⁺ 396; C₂₂H₂₅N₃O₂S requires 395.

### EXAMPLE 1b

### 3-Phenylsulfonyl-8-[(4-dimethylamino)-piperidin-1-yl]-quinoline hydrochloride (E1b)

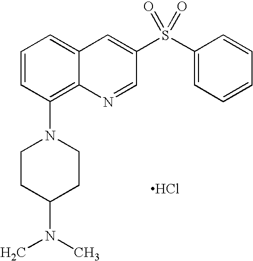

3-Phenylsulfonyl-8-[(4-dimethylamino)-piperidin-1-yl]-quinoline (E1a) was dissolved in methanol and treated with 1M HCl in diethyl ether, then the solvent removed to afford 3-phenylsulfonyl-8-[(4-dimethylamino)-piperidin-1-yl]-quinoline hydrochloride (E1b)

MS: m/z (M+H)⁺ 396; C₂₂H₂₅N₃O₂S requires 395.

### EXAMPLES 2-9 (E2-E9)

Examples 2-9 were prepared from the corresponding alkylamine in place of 4-dimethylamino-piperidine using a method similar to that of Example 1a and the hydrochloride salt prepared by the method of Example 1b:

| Example | Structure | Alkylamine | Mass spectrum |
|---|---|---|---|
| E2 | 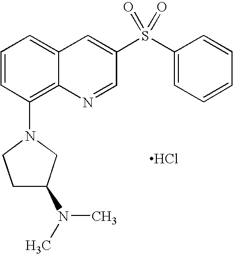 | 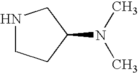 | requires 381; found 382 (MH⁺) |
| E3 | 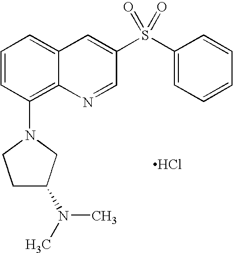 | 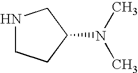 | requires 381; found 382 (MH⁺) |
| E4 | 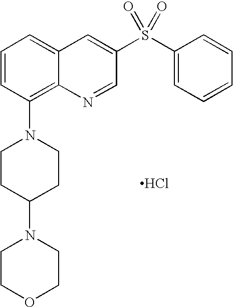 | 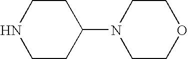 | requires 437; found 438 (MH⁺) |
| E5 | 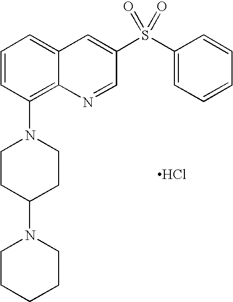 | 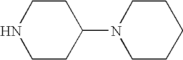 | requires 435; found 436 (MH⁺) |
| E6 | 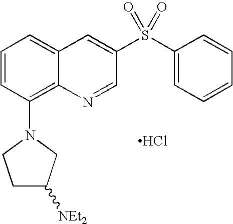 | 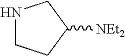 | requires 409; found 410 (MH⁺) |
| E7 | 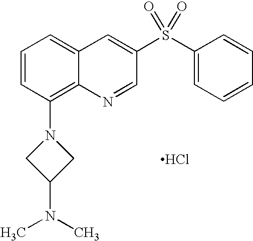 | 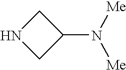 | requires 367; found 368 (MH⁺) |
| E8 | 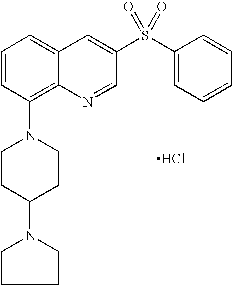 | 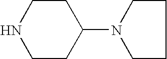 | requires 421; found 422 (MH⁺) |
| E9 | 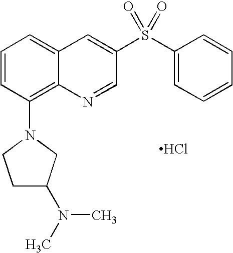 | 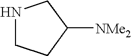 | requires 381; found 382 (MH⁺) |

### EXAMPLE 10

### N-Methyl-1-[3-(phenylsulfonyl)-8-quinolinyl]-4-piperidinamine hydrochloride (E10)

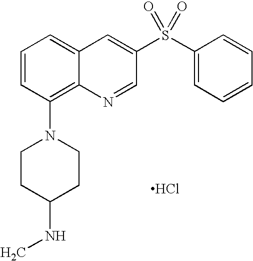

8-Fluoro-3-(phenylsulfonyl)quinoline (D2) (100 mg, 0.35 mmol) and 4-methylamino-piperidine dihydrochloride (112 mg, 0.59 mmol) were suspended in N-methylpyrrolidone (2 ml) and treated with diisopropylethylamine (0.5 ml) then heated to 230° C. under conditions of microwave irradiation for 30 minutes. The mixture was cooled, the solvent evaporated, and the residue purified by preparative HPLC. The free base form was converted to the HCl salt by treatment with 1M HCl in ether followed by evaporation to give N-methyl-1-[3-(phenylsulfonyl)-8-quinolinyl]-4-piperidinamine (E10) as a yellow solid.

MS: m/z (M+H)⁺ 382; C₂₁H₂₃N₃O₂S requires 381.

### EXAMPLE 11

### N-(1-Methylethyl)-1-[3-(phenylsulfonyl)-8-quinolinyl]-4-piperidinamine hydrochloride (E11)

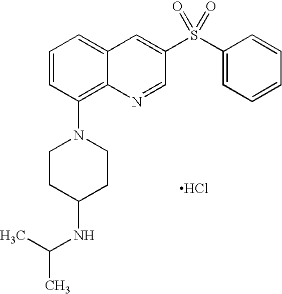

8-Fluoro-3-(phenylsulfonyl)quinoline (D2) (150 mg, 0.52 mmol) and 4-isopropylamino-piperidine dihydrochloride (224 mg, 0.52 mmol) were suspended in N-methylpyrrolidone (2 ml) and treated with diisopropylethylamine (0.5 ml) then heated to 230° C. under conditions of microwave irradiation for 30 minutes. The mixture was cooled, the solvent evaporated, and the residue purified by preparative HPLC. The free base form was converted to the HCl salt by treatment with 1M HCl in ether followed by evaporation to give the N-(1-methylethyl)-1-[3-(phenylsulfonyl)-8-quinolinyl]-4-piperidinamine hydrochloride (E11) as a yellow solid.

MS: m/z (M+H)⁺ 410; C₂₁H₂₃N₃O₂S requires 409.

### EXAMPLE 12

### 1-{3-[(4-Fluorophenyl)sulfonyl]-8-quinolinyl}-N,N-dimethyl-4-piperdinamine hydrochloride (E12)

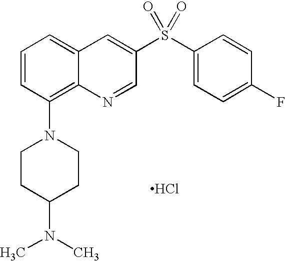

A suspension of 8-chloro-3-[(4-fluorophenyl)sulfonyl]quinoline (D5) (232 mg, 1 equivalent), N,N-dimethyl-4-piperidinamine (139 mg, 1.5 equivalents), tris(dibenzylideneacetone)dipalladium(0) (20.1 mg, 0.03 equivalents), 2-dicyclohexylphosphino-2′-(N,N-dimethylamino)-biphenyl (25.6 mg, 0.09 equivalents) and sodium t-butoxide (104 mg, 1.5 equivalents) was stirred in dioxan (2.5 ml) at 80° C. for 7 hours. The solvent was then evaporated and the reaction mixture dissolved in ethyl acetate and filtered. The mixture was washed with water then brine, dried with magnesium sulphate, filtered and evaporated to dryness, producing a yellow oil. This was then purified on silica eluting with dichloromethane and dichloromethane/methanol/ammonia (79/20/1, 0-50%). 1-{3-[(4-Fluorophenyl)sulfonyl]-8-quinolinyl}-N,N-dimethyl-4-piperidinamine was obtained as a yellow oil (165 mg, 55%). This was then converted to the hydrochloride salt using hydrogen chloride (1M in diethyl ether), and crystallised from ethanol and diethyl ether to afford 1-{3-[(4-fluorophenyl)sulfonyl]-8-quinolinyl}-N,N-dimethyl-4-piperidinamine hydrochloride (E12) as a yellow solid (110 mg).

¹H NMR (CDCl₃) δ2.18 (2H, m), 2.39 (2H, d, J=12 Hz), 2.82 (6H, s), 2.86 (2H, m), 3.32 (1H, m), 4.08 (2H, d, J=12 Hz), 7.24 (2H, t), 2.29 (1H, m), 7.60 (2H, m), 8.04 (2H, m), 8.76 (1H, d, J=2.5 Hz), 9.20 (1H, d, J=2.0 Hz), 12.8 (1H, broad s).

MS: m/z (M+H)⁺ 414; C₂₂H₂₄FN₃O₂S requires 413.

### EXAMPLE 13

### 1-[5-Iodo-3-(phenylsulfonyl)-8-quinolinyl]-N,N-dimethyl-4-piperidinamine hydrochloride (E13)

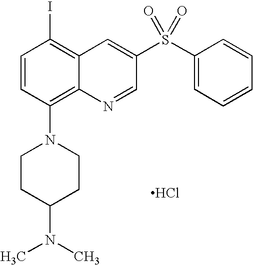

A suspension of N,N-dimethyl-1-[3-(phenylsulfonyl)-8-quinolinyl]-4-piperidinamine hydrochloride (E1b) (200 mg, 0.463 mmol) in acetic acid (0.5 ml) was treated with N-iodosuccinimide (109 mg, 0.486 mmol) and the mixture heated to 50° C. for 14 hours. The cooled mixture was diluted with ethyl acetate (50 ml) and washed with satd. aqueous, sodium sulphite (10 ml) and satd. aqueous sodium bicarbonate (10 ml). The solvent was evaporated and the residue purified by reverse phase mass directed autopreparative chromatography (eluting with MeCN, and aqueous formic acid) then converted to the hydrochloride salt by treatment with 1 M hydrogen chloride in diethyl ether followed by evaporation to obtain 1-[5-iodo-3-(phenylsulfonyl)-8-quinolinyl]-N,N-dimethyl-4-piperidinamine hydrochloride (E13) as a yellow solid (72 mg).

MS: m/z (M+H)⁺ 522; C₂₂H₂₄N₃O₂SI requires 521.

### Pharmacological Data

Compounds of the invention may be tested for in vitro biological activity in accordance with the following cyclase assay:

Cyclase Assay

0.5 μl of test compound in 100% dimethylsulfoxide (DMSO) was added to a white, solid 384 well assay plate (for dose response measurements the top of the concentration range is 7.5 μM final). 10 μl of washed membranes of HeLa 5HT₆ cells (for preparation see WO 98/27081) in basic buffer (50 mM HEPES pH 7.4 (KOH), 10 mM MgCl₂, 100 mM NaCl, 1 μl/ml 3-isobutyl-1-methylxanthine (IBMX) (Sigma-Aldrich)) was added to all wells followed by 10 μl 2×ATP buffer (100 μl/ml ATP and 1 μl/ml 3-Isobutyl-1-methylxanthine (IBMX) (Sigma-Aldrich)) with 5-HT (at a concentration equivalent to a dose response of 4×EC₅₀). The resultant mixture was then incubated at room temperature for 30-45 minutes to allow cAMP production.

cAMP production was then measured using the DiscoveRx™ HitHunter™ chemiluminescence cAMP assay kit (DiscoveRx Corporation, 42501 Albrae Street, Fremont, Calif. 94538; Product Code: 90-0004L) or any other suitable cAMP measurement assay.

IC₅₀ values were estimated from arbitrary designated unit (ADU) measurements from a Perkin Elmer Viewlux instrument using a four parameter logistic curve fit within EXCEL (Bowen, W. P. and Jerman, J. C. (1995), Nonlinear regression using spreadsheets. Trends in Pharmacol. Sci., 16, 413-417). Functional Kᵢ values were calculated using the method of Cheng, Y. C. and Prussof, W. H. (Biochemical Pharmacol (1973) 22 3099-3108). pI₅₀ and fpKᵢ are the negative log10 of the molar IC₅₀ and functional Kᵢ respectively.

The compounds of Examples E1-13 were tested in the above cyclase assay and E1-12 showed antagonist potency for the 5-HT₆ receptor, having fpkᵢ values ≧8.0 at human cloned 5-HT₆ receptors. The fpkᵢ value for Example 13 was 6.8.

1-10. (canceled)

11. A compound of formula (I) or a pharmaceutically acceptable salt thereof: 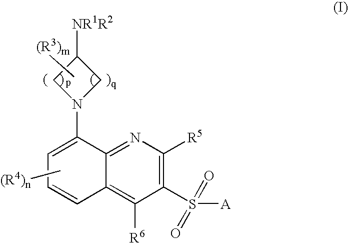
wherein:
R¹ and R² independently represent hydrogen or C₁₋₆ alkyl or R¹ and R² together with the nitrogen atom to which they are attached form a nitrogen containing heterocyclyl group optionally substituted by one or more halogen or C₁₋₆ alkyl groups;
p and q independently represent an integer from 1 to 3;
R³ represents C₁₋₄ alkyl;
m represents an integer from 0 to 4;
R⁴ represents halogen, cyano, —CF₃, CF₃O—, C₁₋₆ alkyl, C₁₋₆ alkoxy, C₁₋₆ alkanoyl or a group —CONR⁷R⁸;
n represents an integer from 0 to 3;
R⁵ and R⁶ independently represent hydrogen, halogen, cyano, —CF₃, CF₃O—, C₁₋₆ alkyl, C₁₋₆ alkoxy, C₁₋₆ alkanoyl or a group —CONR⁷R⁸;
R⁷ and R⁸ independently represent hydrogen or C₁₋₆ alkyl or together with the nitrogen atom to which they are attached form a nitrogen containing heterocyclyl or nitrogen containing heteroaryl group; and
A represents an -aryl, -heteroaryl, -aryl-aryl, -aryl-heteroaryl, -heteroaryl-aryl or -heteroaryl-heteroaryl group;
wherein said aryl and heteroaryl groups of A may be optionally substituted by one or more substituents which may be the same or different, and which are selected from the group consisting of halogen, hydroxy, cyano, nitro, trifluoromethyl, trifluoromethoxy, C₁₋₆ alkyl, trifluoromethanesulfonyloxy, pentafluoroethyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, arylsulfonyl, arylsulfonyloxy, arylsulfonylC₁₋₆ alkyl, C₁₋₆ alkylsulfonamido, C₁₋₆ alkylamido, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, aroylC₁₋₆ alkyl, arylC₁₋₆ alkanoyl, and a group CONR⁹R¹⁰ or SO₂NR⁹R¹⁰, wherein R⁹ and R¹⁰ independently represent hydrogen or C₁₋₆ alkyl or R⁹ and R¹⁰ together with the nitrogen atom to which they are attached form a nitrogen containing heterocyclyl or nitrogen containing heteroaryl group.

12. A compound of formula (I) or a pharmaceutically acceptable salt thereof as defined in claim 11, wherein R¹ and R² independently represent hydrogen or C₁₋₆ alkyl or R¹ and R² together with the nitrogen atom to which they are attached form a pyrrolidinyl, piperidinyl, or morpholinyl ring.

13. A compound of formula (I) or a pharmaceutically acceptable salt thereof as defined in claim 11, wherein A represents aryl optionally substituted by one or more halogen atoms.

14. A compound of formula (I) or a pharmaceutically acceptable salt thereof as defined in claim 11, which is a compound having the formula:

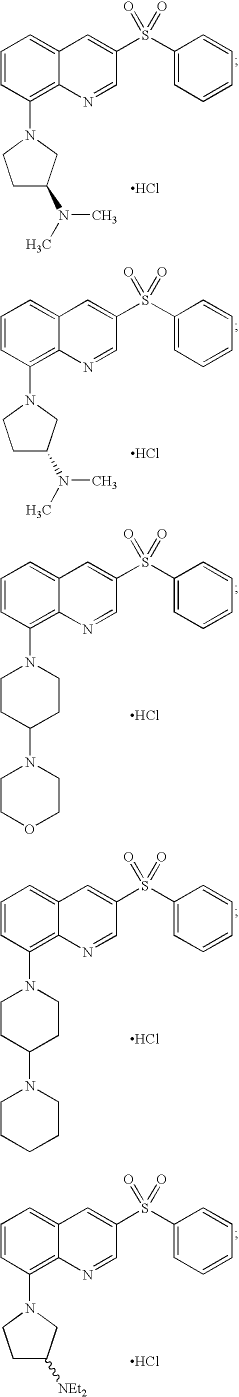

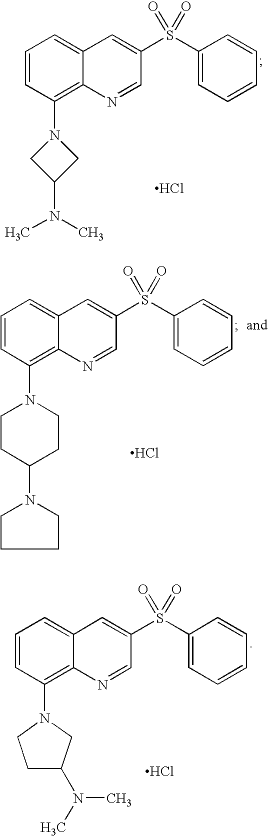

15. A compound of formula (I) or a pharmaceutically acceptable salt thereof as defined in claim 11 selected from:
3-Phenylsulfonyl-8-[(4-dimethylamino)-piperidin-1-yl]-quinoline,
3-Phenylsulfonyl-8-[(4-dimethylamino)-piperidin-1-yl]-quinoline hydrochloride,
N-Methyl-1-[3-(phenylsulfonyl)-8-quinolinyl]-4-piperidinamine hydrochloride,
N-(1-Methylethyl)-1-[3-(phenylsulfonyl)-8-quinolinyl]-4-piperidinamine hydrochloride,
1-{3-[(4-Fluorophenyl)sulfonyl]-8-quinolinyl}-N,N-dimethyl-4-piperidinamine hydrochloride, and
1-[5-Iodo-3-(phenylsulfonyl)-8-quinolinyl]-N,N-dimethyl-4-piperidinamine hydrochloride.

16. A pharmaceutical composition which comprises a compound as defined in claim 11 or a pharmaceutically acceptable salt thereof and a pharmaceutically acceptable carrier or excipient.

17. A method of treating depression, anxiety, Alzheimers disease, age related cognitive decline, ADHD, obesity, mild cognitive impairment, schizophrenia, cognitive deficits in schizophrenia and stroke which comprises administering a safe and therapeutically effective amount to a patient in need thereof of the compound of formula (I) or a pharmaceutically acceptable salt thereof as defined in claim 11.
